SEQUENCE LISTING (8 sequences)

SEQ ID NO: 1 - DNA, 62 bp, Artificial Sequence
  Synthetic primer
atcacngactgnccatanagaggnaagcnnnnnngctttcctctctatgggcagtcggtgat

SEQ ID NO: 2 - DNA, 64 bp, Artificial Sequence
  Synthetic primer
atcgtnacctnagctgantcgganacacgnnnnnncgtgtctccgactcagctaaggtaacgat

SEQ ID NO: 3 - DNA, 31 bp, Artificial Sequence
  Synthetic primer
ccactacgcctccgctttcctctctatgggc

SEQ ID NO: 4 - DNA, 30 bp, Artificial Sequence
  Synthetic primer
ccatctcatccctgcgtgtctccgactcag

SEQ ID NO: 5 - DNA, 43 bp, Artificial Sequence
  Synthetic primer
agatcntcttggnacgatcnnnnngatcgtgccaagaggatct

SEQ ID NO: 6 - DNA, 51 bp, Artificial Sequence
  Synthetic primer
agatcctnttggngtgcgncatcnnnnngatgccgcacgccaagaggatct

SEQ ID NO: 7 - DNA, 57 bp, Artificial Sequence
  Synthetic primer
ccactacgcctccgctttcctctctatgggcagtcggtgatcgtgccaagaggatct

SEQ ID NO: 8 - DNA, 53 bp, Artificial Sequence
  Synthetic primer
ccatctcatccctgcgtgtctccgactcagctaaggtaacgatgccgcacgcc

(19) United States
(12) Patent Application Publication
(10) Pub. No.: US 2016/0257985 A1
(43) Pub. Date: Sep. 8, 2016

(54) DEGRADABLE ADAPTORS FOR BACKGROUND REDUCTION

(71) Applicant: RUBICON GENOMICS, INC., Ann Arbor, MI (US)

(72) Inventors: Emmanuel Kamberov, Ann Arbor, MI (US); John Langmore, Ann Arbor, MI (US); Tim Tesmer, Pinckney, MI (US)

(73) Assignee: RUBICON GENOMICS, INC., Ann Arbor, MI (US)

(21) Appl. No.: 15/035,951

(22) PCT Filed: Nov. 18, 2014
(86) PCT No.: PCT/US2014/066062

§ 371 Date: May 11, 2016

Related U.S. Application Data
(60) Provisional application No. 61/905,546, filed on Nov. 18, 2013.

(51) Int. Cl. C12P 19/34; C12Q 1/68

(52) U.S. Cl. CPC C12P 19/34; C12Q 1/6848; C12Q 1/6806

(57) ABSTRACT

The present disclosure provides systems, processes, articles of manufacture, and compositions that relate to the use of degradable adaptors for background reduction in various nucleic acid manipulations. In particular, adaptors are provided that can be degraded to an extent that the degradation products are incapable or are substantially incapable from participating in subsequent reactions, such as ligation, primer extension, amplification, and sequencing reactions.

This application claims the benefit of U.S. Provisional Patent Application No. 61/905,546, filed Nov. 18, 2013, which is incorporated herein by reference in its entirety.

### BACKGROUND OF THE INVENTION

1. Field of the Invention

The present invention relates generally to the field of molecular biology. More particularly, it concerns preparation and amplification of nucleic acids using degradable adaptors, primers, and other oligonucleotide reagents.

2. Description of Related Art

One problem common to various nucleic acid manipulations, including ligation, amplification, and sequencing reactions, is maintaining a low background of undesired reactions and preventing or reducing the formation of background products. These background reactions and products can result, for example, from contamination, aberrant ligation reactions, primer-dimers, mispriming, and from the use of non-optimal reaction conditions. Oftentimes background products from undesired reactions, or carry-over of unwanted reactants from previous steps hinders or prevents effective analysis of a nucleic acid sample and may preclude further manipulation of the nucleic acid sample. In less severe cases, background can bias analysis of the nucleic acid sample or limit the confidence or accuracy of sequencing results.

In the well-known PCR amplification method, for example, a segment of target DNA having boundaries defined by two oligonucleotide extension primers, or by addition of double-stranded oligonucleotide adaptors to both ends, is exponentially amplified through multiple enzymatic cycles to form additional copies of the target DNA that act as template in successive cycles. A major limitation of PCR lies in the generation of background that includes byproducts formed as a result of amplification of self-ligated adaptor molecules and nonspecific priming events, such as random priming of the nucleic acid template and self-priming of the extension primers. As such, when a high number of amplification cycles are required to amplify a target DNA that is present at a relatively low concentration, the background of nonspecific priming events can significantly impede the effectiveness of PCR amplification and can even prevent subsequent manipulation and analysis of the amplified products.

The presence of background reactions and products resulting from various nucleic acid manipulations can sometimes be overcome by using a separation step prior to detection of a target nucleic acid. In some instances the product of nucleic acid manipulations may include reagents that were intentionally added during one step to manipulate the nucleic acid during that one step; however, those reagents may be detrimental to one or more of the subsequent reactions. With respect to PCR, for example, separation of the amplified target DNA product from the products of nonspecific priming events can be a prerequisite for successful detection and analysis of the amplified target DNA sequence. With respect to PCR, removal of oligonucleotide primers or other oligonucleotides used in a first PCR reaction might be required before adding primers or other oligonucleotides for use in a second PCR reaction. However, using a separation step after one reaction and before a second reaction or assay may decrease the overall efficiency of the process, where reaction yield can suffer, bias or contamination may be introduced into the sample, and overall time and cost increase with respect to analysis of the target nucleic acid or subjecting the target nucleic acid to further manipulation. For example, the separation step may subject the nucleic acid product to molecular loss or contamination produced or introduced during the separation and recovery of the target nucleic acid, impairing various diagnostic nucleic acid analyses of the target nucleic acid. Therefore, in certain instances, it can be preferable to have a reaction in which nucleic acid amplification and detection take place in the same reaction vessel, without the need for background product separation, thereby eliminating the loss of sample due to transfers and inefficient binding and release. During complex molecular procedures, multiple intermediate separation steps might be required before detection, causing multiple losses of samples and delays of results.

### SUMMARY OF THE INVENTION

The present invention allows for the amplification of molecules having at least one double stranded region by using adaptors that avoid the limitations of some adaptor molecules, such as those having the propensity to form amplifiable adaptor dimers. In certain aspects, the present invention provides an inert oligonucleotide for attachment to a double stranded molecule such that it renders the oligonucleotide-ligated molecule capable of being modified, such as amplified, for example by polymerase chain reaction. Upon attachment of the inert adaptor to the molecule, the attached oligonucleotide becomes active and suitable for providing at least in part one or more sequences employable for amplification, while the non-attached, free adaptor and any adaptor dimers are destroyed. As a result, during polymerase chain reaction the free, non-attached inert adaptor and any adaptor dimers can neither be primed nor used as a PCR primer. This provides novel conditions for modification of DNA molecules with the adaptors, and subsequent amplification. These conditions greatly reduce the background in the assay and allow for the use of nanogram, picogram, femtogram, or attogram quantities of input DNA.

In one embodiment, the present invention provides a method for processing a nucleic acid having at least one cleavable base comprising (a) creating an abasic site at the at least one cleavable base; (b) creating a nick at in the backbone of the nucleic acid at the abasic site; and (c) removing at least one nucleotide adjacent to the nick. This method may be used to reduce background resulting from undesired reactions. In some aspects, the at least one nucleotide adjacent to the nick may be 3′ to the nick. In other aspects, the at least one nucleotide adjacent to the nick may be 5′ to the nick. In various aspects, the nucleic acid molecule may be a deoxyribonucleic acid and/or a ribonucleic acid.

In certain aspects of the embodiment, the nucleic acid may comprise a degradable adaptor. For example, the degradable adaptor may be a partially double-stranded oligonucleotide adaptor, a double-stranded oligonucleotide adaptor, or a stem-loop oligonucleotide adaptor. In aspects where the degradable adaptor is a stem-loop oligonucleotide adaptor, the stem-loop oligonucleotide adaptor may comprise (a) a 5′ segment comprising at least one cleavable base; (b) an intermediate segment coupled to the 3′-end of the 5′ segment; and (c) a 3′ segment coupled to the 3′-end of the intermediate segment, wherein the 5′ segment and 3′ segment are at least 80% complementary. In certain aspects, the 5′ segment and 3′ segment may be at least 80%, 85%, 90%, 95%, or 100% complementary. In some aspects, the 3′ segment may not contain a cleavable base. In some aspects, the 5′ segment and the intermediate segment of the stem-loop oligonucleotide adaptor may comprise a cleavable base every 3-6 bases.

In one aspect, the cleavable base may be deoxyuridine. In this aspect, creating an abasic site at the at least one cleavable base may comprise treating the nucleic acid having at least one cleavable base with uracil-DNA glycosylase. In one aspect, creating a nick at the abasic site may comprise treating the nucleic acid comprising an abasic site with an apurinic/apyrimidinic endonuclease (e.g., APE 1). In one aspect, removing at least one nucleotide adjacent to the nick may comprise treating the nucleic acid comprising a nick with an exonuclease (e.g., Exonuclease I).

In some aspects, the method may be a method of processing a nucleic acid used in a first reaction (e.g., degrading a primer used in a first PCR reaction) prior to carrying out a second reaction (e.g., a second PCR reaction, a sequencing reaction, etc.) with a desirable product or component of said first reaction.

In one embodiment, the present invention provides a method for preparing a nucleic acid molecule comprising (a) providing a double stranded nucleic acid molecule; (b) ligating a 3′ end of degradable adaptor comprising at least one cleavable base to a 5′ end of the double stranded nucleic acid molecule to produce an oligonucleotide-attached nucleic acid molecule; (c) creating an abasic site at the at least one cleavable base; (d) creating a nick at the abasic site; and (e) removing at least one nucleotide adjacent to the nick. In one aspect, ligating may produce a nick in the oligonucleotide-attached nucleic acid molecule. In various aspects, the nucleic acid molecule may be a deoxyribonucleic acid and/or a ribonucleic acid. In one aspect, the oligonucleotide-attached nucleic acid molecule may be immobilized (e.g., non-covalently) on a solid support.

In certain aspects of the embodiment, the nucleic acid may comprise a degradable adaptor, which may comprise RNA, DNA, or both. For example, the degradable adaptor may be a partially double-stranded oligonucleotide adaptor, a double-stranded oligonucleotide adaptor, or a stem-loop oligonucleotide adaptor. A stem-loop oligonucleotide may have one or more hairpins. In aspects where the degradable adaptor is a stem-loop oligonucleotide adaptor, the stem-loop oligonucleotide adaptor may comprise (a) a 5′ segment comprising at least one cleavable base; (b) an intermediate segment coupled to the 3′-end of the 5′ segment; and (c) a 3′ segment coupled to the 3′-end of the intermediate segment, wherein the 5′ segment and 3′ segment are at least 80% complementary. In certain aspects, the 5′ segment and 3′ segment may be at least 80%, 85%, 90%, 95%, or 100% complementary. In some aspects, the 3′ segment may not contain a cleavable base. In some aspects, the intermediate segment may comprise at least one cleavable base. In certain aspects, the 5′ segment and the intermediate segment of the stem-loop oligonucleotide adaptor may comprise a cleavable base every 3-6 bases. As such, the adaptor may comprise at least 3, 4, 5, 6 or more cleavable bases depending on the length of the adaptor. In some aspects, the cleavable base may by deoxyuridine. In one aspect, the stem-loop oligonucleotide may comprise a known sequence. In specific aspects, a 5′ end of the stem-loop oligonucleotide lacks a phosphate.

In one aspect of the embodiments, creating an abasic site at the at least one cleavable base may comprise treating the nucleic acid having at least one cleavable base with uracil-DNA glycosylase. In one aspect, creating a nick at the abasic site may comprise treating the nucleic acid comprising an abasic site with an apurinic/apyrimidinic endonuclease. In one aspect, removing at least one nucleotide 3′ to the nick may comprise treating the nucleic acid comprising a nick with an exonuclease. In another aspect, removing at least one nucleotide 5′ to the nick may comprise treating the nucleic acid comprising a nick with an exonuclease. The apurinic/apyrimidinic endonuclease may be APE 1. The exonuclease may be Exonuclease I, Exonuclease III, or lambda exonuclease. In one aspect of the embodiments, the enzymes or chemical treatments must be compatible (e.g., not interfere with) the use of the desirable molecular products either during the cleavage step or in subsequent steps.

In one aspect, a method of the embodiments may comprise amplification of at least part of a processed and/or prepared nucleic acid molecule. Amplification may comprise polymerase chain reaction.

In one aspect, a nucleic acid molecule processed and/or prepared according to the present embodiments may be further modified. For example, the nucleic acid may be subjected to cloning, i.e., incorporation of the modified molecule into a vector. Said incorporation may occur at ends of the modified molecule generated by endonuclease cleavage within an inverted repeat.

In one aspect, a method of the present embodiments may occur in a single suitable solution and/or in the absence of exogenous manipulation. In this aspect, the solution may comprise one or more of a ligase, uracil-DNA glycosylase, an apurinic/apyrimidinic endonuclease, an exonuclease, ATP, and dNTPs. In another aspect, two or more steps of a method of the present embodiments may be performed sequentially.

In one embodiment, there is a kit comprising (a) a nucleic acid comprising at least one cleavable base; (b) a uracil-DNA glycosylase; (c) an apurinic/apyrimidinic endonuclease; and (d) an exonuclease. In one aspect, the apurinic/apyrimidinic endonuclease may be APE 1. In one aspect, the exonuclease may be Exonuclease I or Exonuclease III.

In some aspects, the nucleic acid may comprise a degradable adaptor, which may be a partially double-stranded oligonucleotide adaptor, a double-stranded oligonucleotide adaptor, or a stem-loop oligonucleotide adaptor. In certain aspects, the cleavable base may be deoxyuridine.

In the aspect where the degradable adaptor is a stem-loop oligonucleotide adaptor, the adaptor comprises (a) a 5′ segment comprising at least one cleavable base; (b) an intermediate segment coupled to the 3′-end of the 5′ segment; and (c) a 3′ segment coupled to the 3′-end of the intermediate segment. The 5′ segment and 3′ segment may be at least 80%, 85%, 90%, 95%, or 100% complementary. In one aspect, the 3′ segment may not contain a cleavable base. In one aspect, the intermediate segment may comprise at least one cleavable base. In one aspect, the 5′ segment and the intermediate segment of the stem-loop oligonucleotide adaptor may comprise a cleavable base every 3-6 bases or every 4-5 bases. As such, the adaptor may comprise at least 3, 4, 5, 6 or more cleavable bases depending on the length of the adaptor. In one aspect, the stem-loop oligonucleotide may comprise a known sequence. In specific aspects, a 5′ end of the stem-loop oligonucleotide lacks a phosphate.

Ligating embodiments may be further defined as comprising: generating ligatable ends on the double stranded nucleic acid molecule; generating a ligatable end on the stem-loop oligonucleotide; and ligating one strand of the ligatable end of the stem-loop oligonucleotide to one strand of an end of the nucleic acid molecule, thereby generating a non-covalent junction, such as a nick, a gap, or a 5′ flap structure, in the oligonucleotide-attached nucleic acid molecule. In further aspects, the methods comprise generating blunt ends on the nucleic acid molecule; generating a blunt end on the stem-loop oligonucleotide; and ligating one strand of the blunt end of the stem-loop oligonucleotide to one strand of a blunt end of the nucleic acid molecule, thereby generating a nick in the oligonucleotide-ligated nucleic acid molecule.

Additional embodiments of the invention include a library of DNA molecules prepared by the methods of the invention.

In particular aspects, the present invention is directed to a system and method for preparing a collection of molecules, particularly molecules suitable for amplification, such as amplification utilizing known sequences on the molecules. In specific embodiments, the oligonucleotide comprises a known sequence.

In an additional embodiment, there is a kit housed in a suitable container that comprises one or more compositions of the invention and/or comprises one or more compositions suitable for at least one method of the invention.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Overview of the process of the present technology. (1) Abasic sites are created at cleavable bases (e.g., dU; indicated by circles) in the ligated and free adapter molecules. (2) Nicks are created at the abasic sites. (3) The nucleic acid is degraded at the nick sites.

FIGS. 2A-C—The concerted activities of uracil-DNA glycosylase, apurinic/apyrimidinic (AP) endonuclease, and an exonuclease. FIG. 2A—Samples treated with both APE 1 and Exo I. FIG. 2B—Samples treated with Exo I only. FIG. 2C—Samples treated with APE 1 only.

FIG. 3—Heat-induced degradation of uracil-DNA glycosylase-treated samples.

### DESCRIPTION OF ILLUSTRATIVE EMBODIMENTS

The present disclosure provides systems, processes, articles of manufacture, and compositions that relate to the use of degradable adaptors for background reduction in various nucleic acid manipulations. In particular, adaptors are provided that can be degraded to an extent that the degradation products are incapable or are substantially incapable from participating in subsequent reactions, such as ligation, primer extension, amplification, and sequencing reactions. The degradable adaptors can be partially double-stranded oligonucleotide adaptors, single-stranded oligonucleotide adaptors, stem-loop oligonucleotide adaptors, or any type of oligonucleotide adaptors that may form dimers by ligation and/or primer extension.

The present invention provides several benefits and advantages, which include the following aspects. Degradable adaptors and enzymatic cleavage methods described herein extend the use of cleavable bases in the design of adaptors used for ligation to target nucleic acids beyond simple degradation of the adaptors down to shorter oligonucleotides. In particular, the present technology includes degradation of both non-ligated adaptors and adaptor-dimers down to individual nucleotides. This has a significant impact on the background caused by adaptor-dimers and oligonucleotides released by incomplete adaptor degradation, which allows the use of completely unrelated sequences without the need for suppression caused by terminal inverted repeats. The present technology can be employed as a stand-alone method or in combination with the suppression principle of suppression PCR in amplification of the resulting ligation products. Of note, the methods described herein are distinguishable from methods to reduce background by destruction of oligonucleotides to reduce PCR contamination by unwanted primers by incorporation of deoxyuridine into said primers so that they can later be destroyed using uracil-DNA glycosylase.

Qualitative observations and quantitative experiments show that ligation of a single adaptor or two different adaptors designed to contain a common sequence proximal to the ligation site may have a beneficial effect on the ability to preferentially amplify molecules comprising target inserts of controlled size and discriminate against adaptor dimers carrying no insert or molecules comprising short inserts that have little or no information value. This phenomenon is referred to as suppression or suppression PCR. Suppression refers to the selective exclusion of molecules less than a certain size flanked by terminal inverted repeats, due to their inefficient amplification when the primer(s) used for amplification corresponds) to the entire repeat or a fraction of the repeat (Chenchik et al., 1996; Lukyanov et al., 1999; Siebert et al., 1995; Shagin et al., 1999). The reason for this lies in the equilibrium between productive PCR primer annealing and nonproductive self-annealing of the fragment's complementary ends. At a fixed size of a flanking terminal inverted repeat, the shorter the insert, the stronger the suppression effect and vice versa. Likewise, at a fixed insert size, the longer the terminal inverted repeat, the stronger the suppression effect (Chenchik et al., 1996; Lukyanov et al., 1999; Siebert et al., 1995; Shagin et al., 1999). By virtue of attaching a terminal inverted repeat to both end of a nucleic acid molecule by ligation and/or primer extension one may achieve precise control over the efficiency of primer annealing and extension of target inserts of desired minimal size versus undesirable adaptor dimers or short insert byproducts as described by U.S. Pat. No. 7,803,550.

By way of example, the degradable adaptors can be used in the preparation of nucleic acid libraries, e.g., nucleic acid libraries for massively parallel (NextGen) sequencing, where a target nucleic acid sample is ligated to a stem-loop oligonucleotide adaptor that contains one or more cleavable bases, such as deoxyuracil (dU). Examples of adaptors that can be modified using the present technology include those described in U.S. Pat. No. 8,440,404 to Makarov et al., which is incorporated herein by reference. One can achieve complete degradation or substantially complete degradation of the bulk non-ligated stem-loop oligonucleotide adaptors and any adaptor dimers formed by employing a combination of enzymes in a simultaneous or a sequential fashion to generate abasic sites, create nicks or gaps at the abasic sites, and degrade all or substantially all of the resulting shortened oligonucleotides down to individual nucleotides.

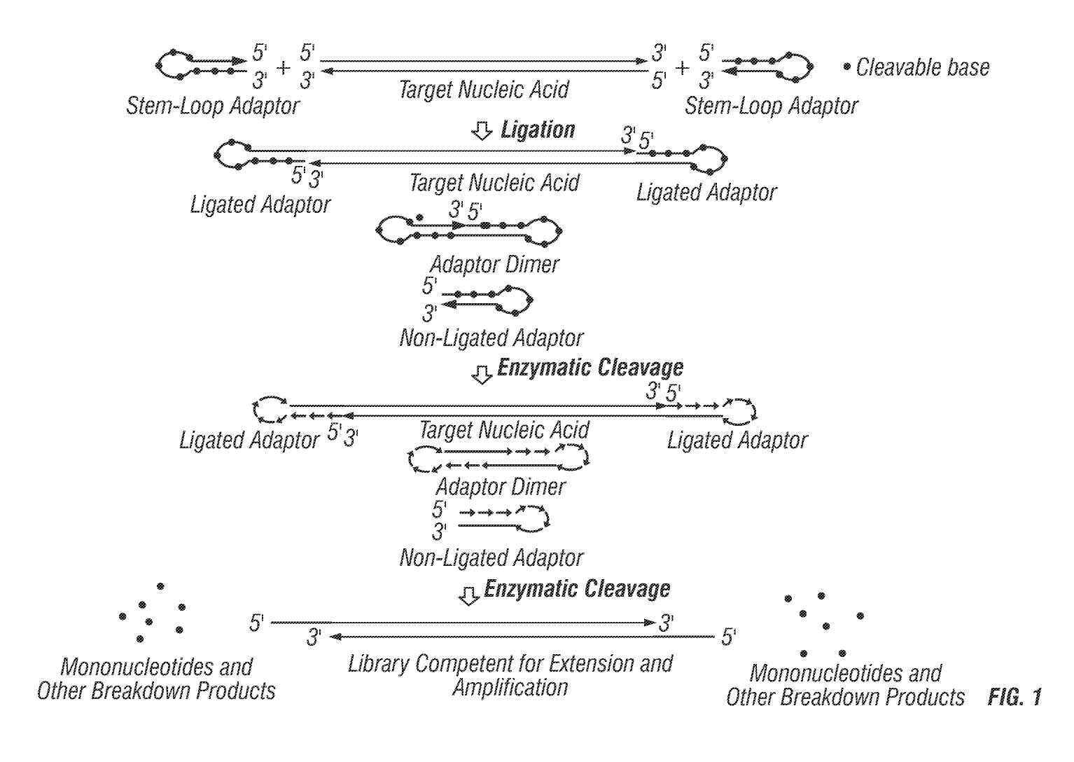

The process can include the following enzymatic steps sequentially or simultaneously (see, FIG. 1):
1) Creating an abasic site at a cleavable base (e.g., dU) using a glycosylase (e.g., uracil-DNA glycosylase (UDG)).
2) Creating a nick at the abasic site using an apurinic/apyrimidinic (AP) endonuclease (e.g., APE 1).
3) Degrading the nucleic acid at the nick site using an exonuclease (e.g., Exo I or Exo III).

With reference to FIG. 1, the 3′-end of a stem-loop adaptor that is ligated to the 5′-end of a target nucleic acid molecule is protected from degradation since it lacks cleavable bases, such as dU, in the resulting ligation product. Following enzymatic cleavage and ligation, the residual 3′-ends of the adaptors can serve as primer binding sites for subsequent amplification or other nucleic acid manipulations. Conversely, adaptor dimers and non-ligated adaptors are degraded following enzymatic cleavage such that they cannot be effectively amplified and cannot participate in various nucleic acid manipulations.

### I. Definitions

“Amplification,” as used herein, refers to any in vitro process for increasing the number of copies of a nucleotide sequence or sequences. Nucleic acid amplification results in the incorporation of nucleotides into DNA or RNA. As used herein, one amplification reaction may consist of many rounds of DNA replication. For example, one PCR reaction may consist of 30-100 “cycles” of denaturation and replication.

“Nucleotide,” as used herein, is a term of art that refers to a base-sugar-phosphate combination. Nucleotides are the monomeric units of nucleic acid polymers, i.e., of DNA and RNA. The term includes ribonucleotide triphosphates, such as rATP, rCTP, rGTP, or rUTP, and deoxyribonucleotide triphosphates, such as dATP, dCTP, dUTP, dGTP, or dTTP.

A “nucleoside” is a base-sugar combination, i.e., a nucleotide lacking a phosphate. It is recognized in the art that there is a certain inter-changeability in usage of the terms nucleoside and nucleotide. For example, the nucleotide deoxyuridine triphosphate, dUTP, is a deoxyribonucleoside triphosphate. After incorporation into DNA, it serves as a DNA monomer, formally being deoxyuridylate, i.e., dUMP or deoxyuridine monophosphate. One may say that one incorporates dUTP into DNA even though there is no dUTP moiety in the resultant DNA. Similarly, one may say that one incorporates deoxyuridine into DNA even though that is only a part of the substrate molecule.

“Incorporating,” as used herein, means becoming part of a nucleic acid polymer.

“Oligonucleotide,” as used herein, refers collectively and interchangeably to two terms of art, “oligonucleotide” and “polynucleotide.” Note that although oligonucleotide and polynucleotide are distinct terms of art, there is no exact dividing line between them and they are used interchangeably herein. The term “adaptor” may also be used interchangeably with the terms “oligonucleotide” and “polynucleotide.”

“Primer” as used herein refers to a single-stranded oligonucleotide or a single- stranded polynucleotide that is extended by covalent addition of nucleotide monomers during amplification. Often, nucleic acid amplification is based on nucleic acid synthesis by a nucleic acid polymerase. Many such polymerases require the presence of a primer that can be extended to initiate nucleic acid synthesis.

The terms “hairpin” and “stem-loop oligonucleotide” as used herein refer to a structure formed by an oligonucleotide comprised of 5′ and 3′ terminal regions, which are inverted repeats that form a double-stranded stem, and a non-self-complementary central region, which forms a single-stranded loop.

The term “in the absence of exogenous manipulation” as used herein refers to there being modification of a DNA molecule without changing the solution in which the DNA molecule is being modified. In specific embodiments, it occurs in the absence of the hand of man or in the absence of a machine that changes solution conditions, which may also be referred to as buffer conditions. However, changes in temperature may occur during the modification.

### II. Cleavable Bases

“Cleavable base,” as used herein, refers to a nucleotide that is generally not found in a sequence of DNA. For most DNA samples, deoxyuridine is an example of a cleavable base. Although the triphosphate form of deoxyuridine, dUTP, is present in living organisms as a metabolic intermediate, it is rarely incorporated into DNA. When dUTP is incorporated into DNA, the resulting deoxyuridine is promptly removed in vivo by normal processes, e.g., processes involving the enzyme uracil-DNA glycosylase (UDG) (U.S. Pat. No. 4,873,192; Duncan, 1981; both references incorporated herein by reference in their entirety). Thus, deoxyuridine occurs rarely or never in natural DNA. Non-limiting examples of other cleavable bases include deoxyinosine, bromodeoxyuridine, 7-methylguanine, 5,6-dihyro-5,6 dihydroxydeoxythymidine, 3-methyldeoxadenosine, etc. (see, Duncan, 1981). Other cleavable bases will be evident to those skilled in the art.

### III. DNA Glycosylase

The term “DNA glycosylase” refers to any enzyme with glycosylase activity that causes excision of a modified nitrogenous heterocyclic component of a nucleotide from a polynucleotide molecule, thereby creating an abasic site.

As used herein, the term “abasic DNA” or “DNA with an abasic site” refers to a DNA molecule, either single-stranded or double-stranded, that contains at least one abasic nucleotide, sometimes called an “abasic site.” An “abasic nucleotide” is a nucleotide that lacks a base in the 1′ position of the deoxyribose.

DNA N-glycosylases include the following enzymes and their homologues in higher eukaryotes, including human homologues: uracil-DNA glycosylase (UDG) and 3-methyladenine DNA glycosylase II (e.g., AlkA) (Nakabeppu et al., 1984; Varshney et al., 1988; Varshney et al., 1991). Additional DNA N-glycosylases include TagI glycosylase and MUG glycosylase (Sakumi et al., 1986; Barrett et al., 1998).

Uracil DNA glycosylases recognize uracils present in single-stranded or double-stranded DNA and cleave the N-glycosidic bond between the uracil base and the deoxyribose of the DNA sugar-phosphate backbone, leaving an abasic site. See, e.g., U.S. Pat. No. 6,713,294. The loss of the uracil creates an apyrimidinic site in the DNA. The enzyme does not, however, cleave the phosphodiester backbone of the DNA molecule.

Uracil-DNA glycosylases, abbreviated as “UDG” or “UNG” include mitochondrial UNG1, nuclear UNG2, SMUG1 (single-strand-selective uracil-DNA glycosylase), TDG (TU mismatch DNA glycosylase), MBD4 (uracil-DNA glycosylase with a methyl-binding domain) and other eukaryotic and prokaryotic enzymes (see, Krokan et al., 2002). An enzyme possessing this activity does not act upon free dUTP, free deoxyuridine, or RNA (Duncan, 1981).

An additional example of UDG enzymes for creating one or more abasic sites is a thermostable homolog of the E. coli UDG from Archaeoglobus fulgidus. Afu UDG catalyzes the release of free uracil from uracil-containing DNA. Afu UDG efficiently hydrolyzes uracil from single-stranded or double-stranded DNA. Another example includes Antarctic thermolabile UDG, which catalyzes the release of free uracil from uracil-containing single-stranded or double-stranded DNA. The Antarctic thermolabile UDG enzyme is sensitive to heat and can be rapidly and completely inactivated at temperatures above 50° C.

Non-limiting examples of additional cleavable bases and their respective nicking agents are as follows: AlkA glycosylase recognizes and cleaves deoxyinosine residues; DNA-7-methylguanine glycosylases recognize and cleave 7-methylguanine residues; hypoxanthine-NDA glycosylase recognizes and cleaves hypoxanthine residues; 3-methyladenine-DNA glycosylase I (e.g., TagI) and 3-methyladenine-DNA glycosylase II (e.g., AlkA) recognize and cleave 3-methyladenine residues; Fpg recognizes and cleaves 8-oxo-guanine residues; and Mug recognizes and cleaves 3,N(4)-ethenocytosine and uracil residues from DNA.

### IV. Apurinic/apyrimidinic Endonuclease

As used herein, the term “AP endonuclease” or “AP lyase” means an enzyme capable of breaking a phosphodiester backbone of a nucleic acid at an abasic site. The term includes the enzymes capable of breaking the backbone both 5′ and 3′ of the abasic site.

The DNA sugar-phosphate backbone that remains after, for example, UDG cleavage of the glycosidic bond can then be cleaved, for example, by alkaline hydrolysis, elevated temperature, tripeptides containing aromatic residues between basic ones, such as Lys-Trp-Lys and Lys-Tyr-Lys (Pierre et al., 1981; Doetsch et al., 1990), and AP endonucleases, such as endonuclease IV, endonuclease V, endonuclease III, endonuclease VI, endonuclease VII, human endonuclease II, and the like. Therefore, an enzyme such as APE I may be used in conjunction with UDG to remove dU resides from and then nick a nucleic acid molecule.

Examples of enzymes for creating a nick at an abasic site include apurinic/apyrimidinic (AP) endonucleases, such as APE 1 (also known as HAP 1 or Ref-1), which shares homology with E. coli exonuclease III protein. APE 1 cleaves the phosphodiester backbone immediately 5′ to an AP site, via a hydrolytic mechanism, to generate a single-strand DNA break leaving a 3′-hydroxyl and 5′-deoxyribose phosphate terminus.

An artificial nicking agent may be created by combining a DNA N-glycosylase and an AP endonuclease, for example by combining UDG glycosylase with APE I endonuclease or AlkA glycosylase with EndoIV endonuclease to achieve single-stranded cleavage at a modified nucleotide.

In certain embodiments of the invention, different types of modified nucleotides may be introduced at a plurality of selected locations in order to nick target molecule(s) sequentially at two or more locations. For example, a deoxyuridine, an 8-oxo-guanine, and a deoxyinosine may be introduced into the selected locations of the target molecule(s). A single nicking agent may be formulated that includes more than one specificity component according to the incorporated modified nucleotides. Alternatively separate nicking agents may be formulated and applied to the target molecule(s) sequentially. For example, AlkA and FPG glycosylase/AP lyase, which selectively nicks at a deoxyinosine and deoxy 8-oxo-guanine may be combined or used sequentially with a nicking agent that contains UDG and EndoVIII glycosylase/AP lyase that selectively nicks at a deoxyuridine.

Examples of nicking agents described herein that are capable of excising modified nucleotides include: for excising deoxyuridine—UDG glycosylase in a mixture with EndoIV endonuclease; UDG glycosylase in a mixture with FPG glycosylase/AP lyase; UDG glycosylase in a mixture with EndoVIII glycosylase/AP lyase; a mixture containing UDG glycosylase, EndoIV endonuclease and EndoVIII glycosylase/AP lysase; for excising 8-oxo-guanine and deoxyuridine—a mixture containing UDG glycosylase, FPG glycosylase/AP lyase and EndoIV endonuclease or UDG glycosylase in a mixture with FPG glycosylase/AP lyase; and for excising deoxyinosin—AlkA glycosylase in a mixture with EndoVIII glycosylase/AP lyase or AlkA glycosylase in a mixture with FPG glycosylase/AP lyase.

Endonuclease VIII from E. coli acts as both an N-glycosylase and an AP-lyase. The N-glycosylase activity releases degraded pyrimidines from double-stranded DNA, generating an AP site. The AP-lyase activity cleaves 3′ to the AP site leaving a 5′ phosphate and a 3′ phosphate. Degraded bases recognized and removed by Endonuclease VIII include urea, 5,6-dihydroxythymine, thymine glycol, 5-hydroxy-5-methylhydantoin, uracil glycol, 6-hydroxy-5,6-dihydrothymine and methyltartronylurea. While Endonuclease VIII is similar to Endonuclease III, Endonuclease VIII has β and δ lyase activity while Endonuclease III has β lyase activity.

Fpg (formamidopyrimidine [fapy]-DNA glycosylase) (also known as 8-oxoguanine DNA glycosylase) acts both as an N-glycosylase and an AP lyase. The N-glycosylase activity releases degraded purines from double stranded DNA, generating an apurinic (AP site). The AP lyase activity cleaves both 3′ and 5′ to the AP site thereby removing the AP site and leaving a one base gap. Some of the degraded bases recognized and removed by Fpg include 7,8-dihydro-8-oxoguanine (8-oxoguanine), 8-oxoadenine, fapy-guanine, methyl-fapy-guanine, fapy-adenine, aflatoxin Bl-fapy-guanine, 5-hydroxy-cytosine and 5-hydroxy-uracil.

Also contemplated are the nicking agents referred to as the USER™ Enzyme, which specifically nicks target molecules at deoxyuridine, and the USER™ Enzyme 2, which specifically nicks target molecules at both deoxyuridine and 8-oxo-guanine both leaving a 5′ phosphate at the nick location (see, U.S. Pat. No. 7,435,572). USER™ Enzyme is a mixture of uracil-DNA glycosylase (UDG) and the DNA glycosylase-lyase Endonuclease VIII. UDG catalyzes the excision of a uracil base, forming an abasic (apyrimidinic) site while leaving the phosphodiester backbone intact. The lyase activity of Endonuclease VIII breaks the phosphodiester backbone at the 3′ and 5′ sides of the abasic site so that base-free deoxyribose is released.

### V. Exonuclease

Examples of enzymes for degrading a nucleic acid at a nick site include various exonucleases, such as Exonuclease I (Exo I) and Exonuclease III (Exo III). Exo I (E. coli) catalyzes the removal of nucleotides from single-stranded DNA in the 3′ to 5′ direction. For example, Exo I can degrade single-stranded oligonucleotides in a reaction mixture containing double-stranded nucleic acid products. Exo III (E. coli) catalyzes the stepwise removal of mononucleotides from 3′-hydroxy termini of duplex DNA. A limited number of nucleotides are removed during each binding event, resulting in coordinated progressive deletions within the population of DNA molecules. The preferred substrates are blunt or recessed 3′ termini, although the enzyme also acts at nicks in duplex DNA to produce single-strand gaps. Lambda exonuclease may be used to enzymatically degrade a nucleic acid at a nicked site in a 5′ to 3′ direction.

### VI. Adaptors and Their Use for DNA Processing

Supplementing DNA ends with additional short polynucleotide sequences, referred to as adaptors or linkers, is used in many areas of molecular biology. The usefulness of adapted DNA molecules is illustrated by, but not limited to, several examples, such as ligation-mediated locus-specific PCR, ligation-mediated whole genome amplification, adaptor-mediated DNA cloning, DNA affinity tagging, DNA labeling, etc.

### A. Ligation-Mediated Amplification of Unknown Regions Flanking a Known DNA Sequence

Libraries generated by DNA fragmentation and addition of a universal adaptor to one or both DNA ends were used to amplify (by PCR) and sequence DNA regions adjacent to a previously established DNA sequence (see, for example, U.S. Pat. No. 6,777,187 and references therein, all of which are incorporated by reference herein in their entirety). The adaptor can be ligated to the 5′ end, the 3′ end, or both strands of DNA. The adaptor can have a 3′ or 5′ overhang. It can also have a blunt end, especially in the cases when DNA ends are “polished” after enzymatic, mechanical, or chemical DNA fragmentation. Ligation-mediated PCR amplification is achieved by using a locus-specific primer (or several nested primers) and a universal primer complementary to the adaptor sequence.

### B. Ligation-Mediated Whole Genome Amplification

Libraries generated by DNA fragmentation and subsequent attachment of a universal adaptor to both DNA ends were used to amplify whole genomic DNA (whole genome amplification, or WGA) (see, for example, U.S. Pat. Publn. No. 2004/0209299 and U.S. Pat. No. 7,718,403 and references therein, all of which are incorporated by reference herein in their entirety). The adaptor can be ligated to both strands of DNA or only to the 3′ end followed by extension. The adaptor can have a 3′ or 5′ overhang, depending on the structure of the DNA end generated by the restriction enzyme or other enzyme used to digest DNA. It can also have a blunt end, such as in the cases where DNA ends after enzymatic DNA cleavage are blunt or when the ends are repaired and “polished” after enzymatic, mechanical, or chemical DNA fragmentation. Whole genome PCR amplification is achieved by using one or two universal primers complementary to the adaptor sequence(s), in specific embodiments.

### C. Adaptor-Mediated DNA Cloning

Adaptors (or linkers) are frequently used for DNA cloning (see, for example, Sambrook et al., 1989). Ligation of double stranded adaptors to DNA fragments produced by sonication, nebulization, or hydro-shearing process followed by restriction digestion within the adaptors allows production of DNA fragments with 3′ or 5′ protruding ends that can be efficiently introduced into a vector sequence and cloned.

### VII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Degradable Adaptors for Background Reduction—Heat Degradation

The following example illustrates the use of degradable adaptors comprising degradable abasic sites (dU) in the non-ligated strand to allow degradation of free adaptors and adaptor dimers down to small oligonucleotides using heat-induced degradation.

Template Preparation. Ten microliters of each DNA sample (200 pg Covaris-sheared human gDNA) was added to a PCR plate well. For non-template controls (NTC), 10 μL of nuclease-free water was substituted for the DNA sample. A pre-mix of 2 μL/sample Template Preparation Buffer ((6.5× ATP-free ligase buffer comprising: 325 mM Tris-HCl pH 7.6 @ 25° C., 65 mM MgCl₂, 3.25 mM DTT) supplemented with dNTP mix (2.5 mM each dNTP)) and 1 μL/sample Template Preparation Enzyme (End Repair Mix, Enzymatics Cat # Y914-LC-L) was prepared in a separate tube and mixed by pipette. Then, 3 μL of the pre-mix was added to the 10 μL DNA sample in the PCR tube or well and mixed 4-5 times was a pipette set to 8 μL. The final concentration of the reaction components was as follows: 50 mM Tris-HCl pH 7.6 @ 25° C., 10 mM MgCl₂, 0.5 mM DTT, 385 μM dNTPs, 1× End Repair Enzymes. The PCR plate was centrifuged and incubated in a thermal cycler using the following conditions: 1 cycle at 22° C. for 25 min; 1 cycle at 55° C. for 20 min; hold at 22° C.

Library Synthesis. Fresh Library Synthesis pre-mix of 1 μL/sample Library Synthesis Buffer (2× ATP-free ligase buffer comprising: 100 mM Tris-HCl pH 7.6 @ 25° C., 20 mM MgCl₂, 1.0 mM DTT supplemented with 15 mM ATP and 15 μM each stem-loop adaptor oligo—Table 1; SEQ ID NOs: 5 and 6) and 1 μL/sample Library Synthesis Enzyme Mix (comprising: 1.2 U Uracil DNA Glycosylase (UDG, Enzymatics # G5010L) and 8 U T4 DNA Ligase (Enzymatics # L603-HC-L) per μL) was prepared in a separate tube and mixed by pipette. Then, 2 μL of the Library Synthesis pre-mix were added to each sample and mixed 4-5 times with a pipette set to 10 μL. The final concentration of the reaction components was as follows: 50 mM Tris-HCl pH 7.6 @ 25° C., 10 mM MgCl₂, 0.5 mM DTT, 334 μM dNTPs, 1 mM ATP, 1.2 U Uracil DNA Glycosylase, 8 U T4 DNA Ligase, 1 μM each adaptor oligo. The plate was centrifuged and incubated in a thermal cycler using the following conditions: 1 cycle at 22° C. for 40 min; hold at 4° C.

ThruPLEX-FD Library Amplification. Library Amplification pre-mix of 4.25 μL/sample nuclease-free water, 3.75 μL/sample EvaGreen®:fluorescein (FC; 9:1), and 50.5 μL/sample Library Amplification Buffer (comprising: 150 mM Tris-SO₄, pH 8.5 @ 25° C., 120 mM TMAC, 0.75 mM MgCl₂, 0.06% w/v Gelatin, supplemented with 0.375 μM of each PCR oligo—Table 1; SEQ ID NOs: 7 and 8) was prepared in a separate tube immediately prior to use.

For samples to be heated after polymerase addition, 1.5 μL/sample Library Amplification Enzyme (KAPA HiFi™ DNA Polymerase (KK2102) at 1 U/μl) was added to the pre-mix. Then, 60 μL of the Library Amplification pre-mix was added to each library and mixed 3-4 times with a pipette set to 60 μL.

For samples to be heated prior to polymerase addition, 58.5 μL/sample Library Amplification pre-mix without KAPA HiFi™ DNA Polymerase was added to each library and mixed 3-4 times with a pipette set to 60 μL. The samples were heated for 5 min at 85° C., and then 1.5 μL Library Amplification Enzyme (KAPA HiFi™ DNA Polymerase (KK2102) at 1 U/μL) was added to each sample.

For all reactions, the final concentration of the reaction components was as follows: 100 mM Tris-SO₄, pH 8.5 @ 25° C., 80 mM TMAC, 2.5 mM MgCl₂, 0.04% w/v Gelatin, 1× EvaGreen® fluorescent reporter dye, 1× calibration dye (fluorescein), 1.5 U KAPA HiFi™ DNA Polymerase, 0.25 μM each PCR oligo. The plates were centrifuged and then incubated in a real-time thermal cycler as follows: 1 cycle at 72° C. for 3 min; 1 cycle at 85° C. for 2 min; 1 cycle at 98° C. for 2 min; 4 cycles of 98° C. for 20 sec, 67° C. for 20 sec, 72° C. for 40 sec; and 4-21 cycles of 98° C. for 20 sec and 72° C. for 50 sec.

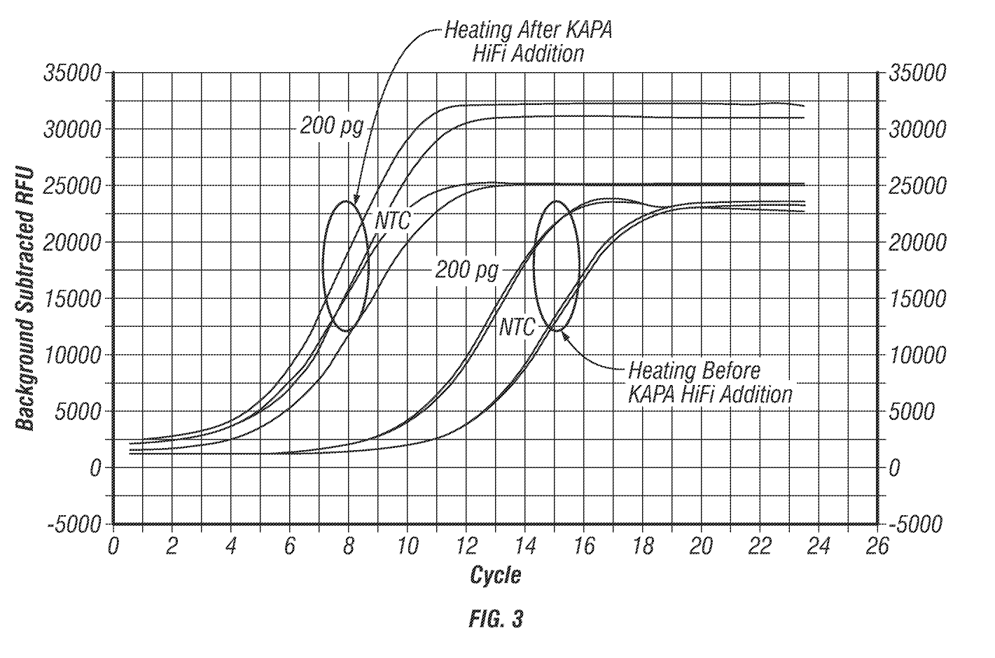

Conclusion. Heat degradation of adaptors and adaptor dimers resulted in about a 6.5 cycle (100-fold) right shift and improved signal-to-noise ratio (FIG. 3).

### Example 2—Degradable Adaptors for Background Reduction—Enzymatic Degradation

The following example illustrates the surprising, unexpected, and synergistic effects between the combined enzymatic activities used in the present degradable adaptor technology, i.e., the concerted activities of uracil-DNA glycosylase, apurinic/apyrimidinic (AP) endonuclease, and an exonuclease.

Pooled human lymphocyte DNA from healthy donors was diluted to 23.8 pg/μL in TE buffer and subjected to simultaneous fragmentation and end-repair. Ten microliter aliquots of diluted DNA or no template controls (NTC) containing TE buffer were supplemented with NEBNext® dsDNA Fragmentase® Reaction Buffer comprising 20 mM Tris-HCl, 50 mM NaCl, 10 mM MgCl₂, 0.15% Triton® X-100, pH 7.5 @ 25° C., in a final volume of 13 μL containing 1 μL of NEBNext® dsDNA Fragmentase® (New England Biolabs, Cat # M0348S) and 0.5 μL of End-Repair Mix (Enzymatics Cat # Y9140-LC-L). Samples were incubated for 30 min at 22° C., followed by 20 min at 55° C. and 2 min at 22° C.

Next, a mixture of stem-loop oligonucleotide adaptors (Table 1, SEQ ID NOs: 1 and 2) each at a 1 μM final concentration, 240 U of T4 DNA Ligase (Enzymatics Cat # L6030-HC) and 6 U of uracil-DNA glycosylase (Enzymatics Cat # G5010L) were added to each sample to a final volume of 15 μL and the samples were incubated for 40 min at 22° C., followed by 15 min for 55° C. and 2 min at 37° C.

To test the degradation of free adaptor molecules and adaptor dimers, 15 U of human apurinic/apyrimidinic (AP) endonuclease, APE 1 (New England Biolabs Cat # M0282S), or 10 U of E. coli Exo I (New England Biolabs Cat # M0293S) were added to DNA-containing samples or NTC controls and incubated for 15 min at 37° C., 3 min at 42° C., 3 min at 45° C., and 10 min at 55° C. Controls containing both APE 1 and Exo I were also run in parallel in order to interrogate potential synergistic effects of the nucleases.

To amplify the libraries, 60 μL of PCR master mix comprising 1× KAPA HiFi™ DNA Polymerase Fidelity Buffer, 1.5 U of KAPA HiFi™ DNA Polymerase (KAPA Biosystems Cat # KK2101), 1× EvaGreen® fluorescent reporter dye (Biotium, Inc. Cat # 31000), 1× calibration dye (fluorescein), 0.3 mM dNTP mix, and 0.35 μM of each PCR primer (Table 1, SEQ ID NOs: 3 and 4) were added to all samples and NTC controls. Amplification was carried out using a BioRad iCycler™ real-time PCR instrument with the following cycling protocol: 1 cycle at 72° C. for 3 min; 1 cycle at 85° C. for 2 min; 1 cycle at 98° C. for 2 min; 4 cycles at 98° C. for 20 sec, 65° C. for 20 sec, and 72° C. for 40 sec; and 25 cycles at 98° C. for 20 sec and 72° C. for 50 sec. Real-time data was acquired at the 72° C. extension step of the last 25 cycles.

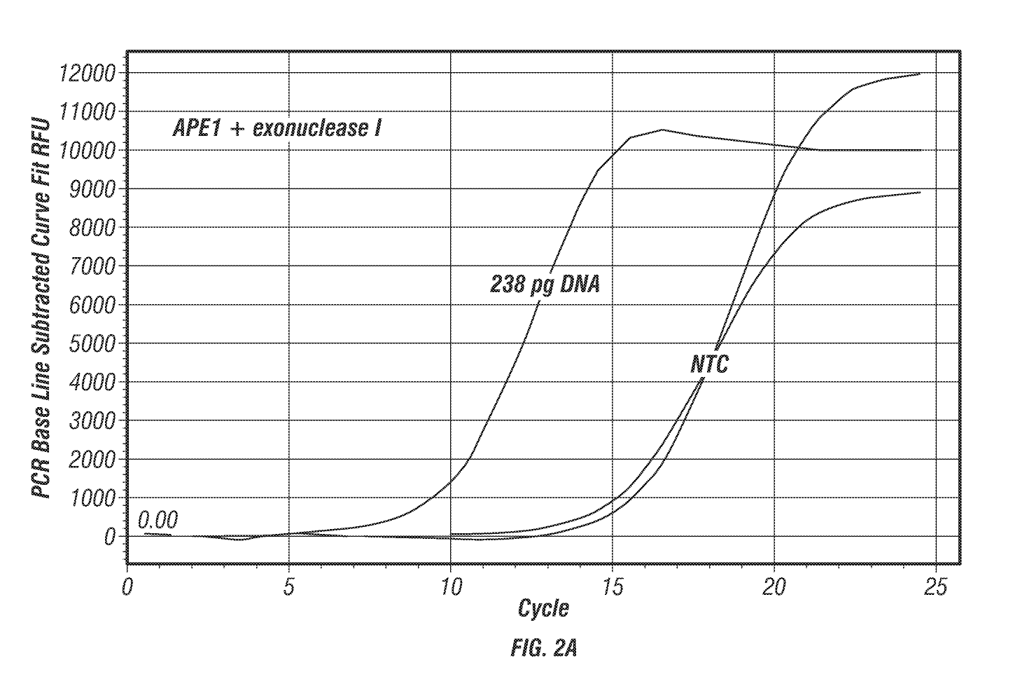

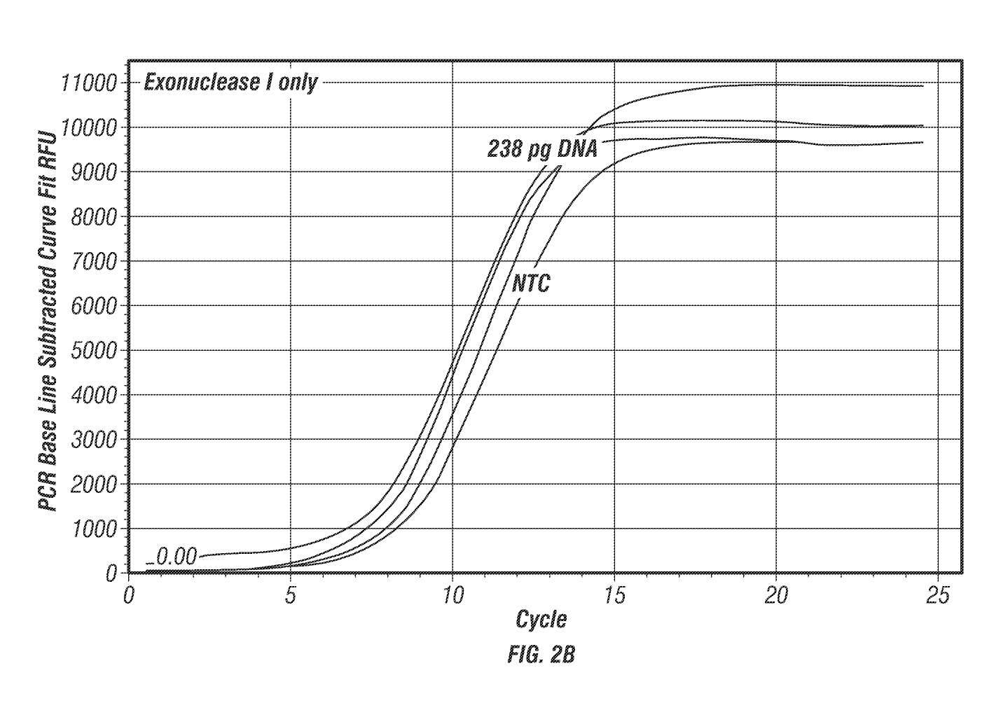

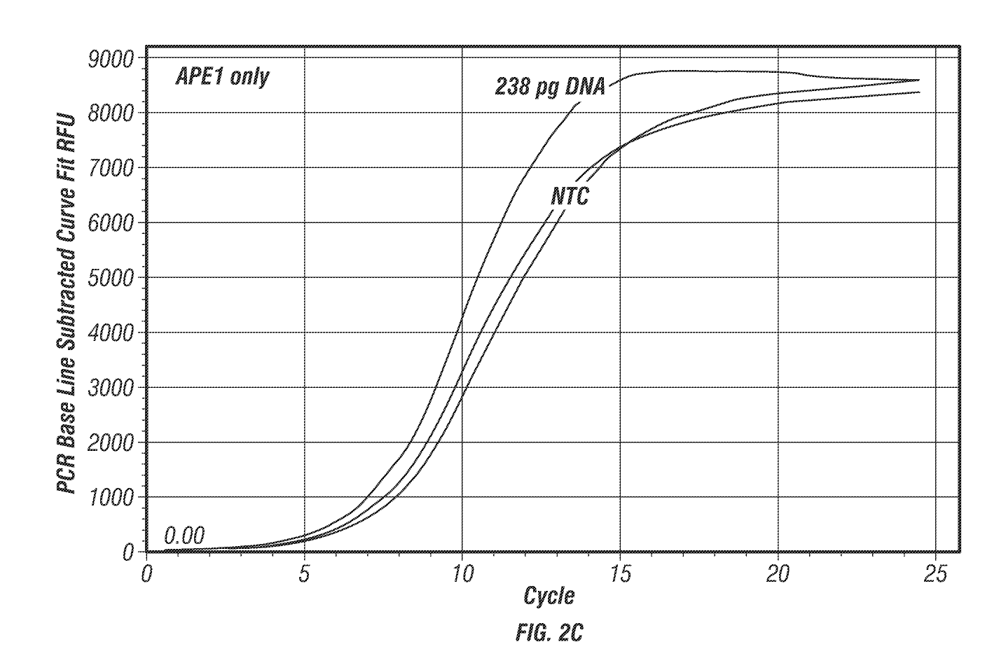

As shown in FIG. 2A, the simultaneous presence of APE 1 and Exo I resulted in a greater than 5-cycle right shift (>32-fold decrease) of the background caused by adaptor dimers, whereas none of the individual nucleases were capable of significantly degrading the dimers resulting from ligation of two adaptor molecules to each other (FIGS. 2B and 2C).

TABLE 1
Oligonucleotide sequences.

| SEQ | |
|---|---|
| ID | |
| NO | Oligonucleotide |
| 1 | 5′-ATCACUGACTGUCCATAUAGAGGUAAGCUUUUUUGCTTTCCTCT |
| | CTATGGGCAGTCGGTGAT-3′ |
| 2 | 5′-ATCGTUACCTUAGCTGAUTCGGAUACACGUUUUUUCGTGTCTCC |
| | GACTCAGCTAAGGTAACGAT-3′ |
| 3 | 5′-CCACTACGCCTCCGCTTTCCTCTCTATGGGC-3′ |
| 4 | 5′-CCATCTCATCCCTGCGTGTCTCCGACTCAG-3′ |
| 5 | 5′-AGATCUTCTTGGUACGATCUUUUUGATCGTGCCAAGAGGATCT-3′ |
| 6 | 5′-AGATCCTUTTGGUGTGCGUCATCUUUUUGATGCCGCACGCCAAG |
| | AGGATCT-3′ |
| 7 | 5′-CCACTACGCCTCCGCTTTCCTCTCTATGGGCAGTCGGTGATCGTG |
| | CCAAGAGGATCT-3′ |
| 8 | 5′-CCATCTCATCCCTGCGTGTCTCCGACTCAGCTAAGGTAACGATGC |
| | CGCACGCC-3′ |

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

U.S. Pat. No. 4,873,192

U.S. Pat. No. 6,713,294

U.S. Pat. No. 6,777,187

U.S. Pat. No. 7,435,572

U.S. Pat. No. 7,718,403

U.S. Pat. No. 7,803,550

U.S. Pat. No. 8,440,404

U.S. Pat. Publn. No. 2004/0209299

Barrett et al., Crystal structure of a G:T/U mismatch-specific DNA glycosylase: mismatch recognition by complementary-strand interactions, Cell, 92:117-129, 1998.

Chenchik et al., Full-length cDNA cloning and determination of mRNA 5′ and 3′ ends by amplification of adaptor-ligated cDNA, Biotechniques, 21:526-534, 1996.

Doetsch et al., The enzymology of apurinic/apyrimidinic endonucleases, Mutation Research, 236:173-201, 1990.

Duncan, DNA Glycosylases, In: The Enzymes, XIV:565-586, 1981.

Krokan et al., Uracil in DNA - occurrence, consequences and repair, Oncogene, 21:8935-9232, 2002.

Lukyanov et al., Selective suppression of polymerase chain reaction, Bioorganicheskaya Khimiya, 25:163-170, 1999.

Nakabeppu et al., loning and characterization of the alkA gene of Escherichia coli that encodes 3-methyladenine DNA glycosylase II, J. Biol. Chem., 259:13723-13729, 1984.

Pierre et al., Specific nicking of DNA at apurinic sites by peptides containing aromatic residues, J. Biol. Chem., 256:10217-10226, 1981.

Sakumi et al., Purification and structure of 3-methyladenine-DNA glycosylase I of Escherichia coli, J. Biol. Chem., 261:15761-15766, 1986.

Sambrook et al., Molecular Cloning: a laboratory manual. 2nd ed. N.Y., Cold Spring Harbor Laboratory, Cold Spring Harbor Laboratory Press, 1989.

Shagin et al., Regulation of average length of complex PCR product, Nucleic Acids Research, 27, e23, 1999.

Siebert et al., An Improved PCR Method for Walking in Uncloned Genomic DNA, Nucleic Acids Research, 23:1087-1088, 1995.

Varshney et al., Sequence analysis, expression and conservation of Escherichia coli uracil DNA glycosylase and its gene (ung), J. Biol. Chem., 263:7776-7784, 1988.

Varshney et al., Specificities and kinetics of uracil excision from uracil-containing DNA oligomers by Escherichia coli uracil DNA glycosylase, Biochemistry, 30:4055-4061, 1991.

What is claimed is:

1. A method for processing a nucleic acid having at least one cleavable base, comprising:
(a) creating an abasic site at the at least one cleavable base;
(b) creating a nick in the backbone of the nucleic acid at the abasic site; and
(c) removing at least one nucleotide adjacent to the nick.

2. The method of claim 1, wherein the nucleic acid comprises a degradable adaptor.

3. The method of claim 2, wherein the degradable adaptor is a partially double-stranded oligonucleotide adaptor, a double-stranded oligonucleotide adaptor, or a stem-loop oligonucleotide adaptor.

4. The method of claim 3, wherein the stem-loop oligonucleotide adaptor comprises:
(a) a 5′ segment comprising at least one cleavable base;
(b) an intermediate segment coupled to the 3′-end of the 5′ segment; and
(c) a 3′ segment coupled to the 3′-end of the intermediate segment, wherein the 5′ segment and 3′ segment are at least 80% complementary.

5. The method of claim 4, wherein the 3′ segment does not contain a cleavable base.

6. The method of claim 4, wherein the intermediate segment comprises at least one cleavable base.

7. The method of claim 4, wherein the 5′ segment and the intermediate segment of the stem-loop oligonucleotide adaptor comprises a cleavable base every 3-6 bases.

8. The method of any one of claims 1 and 4-7, wherein the cleavable base is deoxyuridine.

9. The method of claim 1, wherein creating an abasic site at the at least one cleavable base comprises treating the nucleic acid having at least one cleavable base with uracil-DNA glycosylase.

10. The method of claim 1, wherein creating a nick at the abasic site comprises treating the nucleic acid of step (a) with an apurinic/apyrimidinic endonuclease.

11. The method of claim 1, wherein removing at least one nucleotide adjacent to the nick comprises treating the nucleic acid of step (b) with an exonuclease.

12. The method of claim 10, wherein the apurinic/apyrimidinic endonuclease is APE 1.

13. The method of claim 11, wherein the exonuclease is Exonuclease I.

14. A method for preparing a nucleic acid molecule, comprising:
(a) providing a double stranded nucleic acid molecule;
(b) ligating a 3′ end of degradable adaptor comprising at least one cleavable base to a 5′ end of the double stranded nucleic acid molecule to produce an oligonucleotide-attached nucleic acid molecule;
(c) creating an abasic site at the at least one cleavable base;
(d) creating a nick at the abasic site; and
(e) removing at least one nucleotide adjacent to the nick.

15. The method of claim 14, wherein the degradable adaptor is a partially double-stranded oligonucleotide adaptor, a double-stranded oligonucleotide adaptor, or a stem-loop oligonucleotide adaptor.

16. The method of claim 15, wherein the stem-loop oligonucleotide adaptor comprises:
(i) a 5′ segment comprising at least one cleavable base;
(ii) an intermediate segment coupled to a 3′-end of the 5′ segment; and
(iii) a 3′ segment coupled to a 3′-end of the intermediate segment, wherein the 5′ segment and the 3′ segment are at least 80% complementary.

17. The method of claim 16, wherein the 3′ segment does not contain a cleavable base.

18. The method of claim 15, wherein the intermediate segment comprises at least one cleavable base.

19. The method of claim 18, wherein the 5′ segment and the intermediate segment of the stem-loop oligonucleotide adaptor comprises a cleavable base every 3-6 bases.

20. The method of any one of claims 14 and 16-19, wherein the cleavable base is deoxyuridine.

21. The method of claim 14, wherein the ligating produces a nick in the oligonucleotide-attached nucleic acid molecule.

22. The method of claim 14, wherein the double stranded nucleic acid molecule is a double stranded DNA molecule.

23. The method of claim 14, further comprising amplification of at least part of the oligonucleotide-attached nucleic acid molecule.

24. The method of claim 23, wherein the amplification comprises polymerase chain reaction.

25. The method of claim 16, wherein the stem-loop oligonucleotide comprises a known sequence.

26. The method of claim 14, wherein the oligonucleotide-attached nucleic acid molecule is further modified.

27. The method of claim 26, wherein the further modification comprises cloning.

28. The method of claim 27, wherein cloning is further defined as comprising incorporation of the modified molecule into a vector, said incorporation occurring at ends in the modified molecule generated by endonuclease cleavage within the inverted repeat.

29. The method of claim 14, wherein the method is further defined as occurring in a single suitable solution, wherein the process occurs in the absence of exogenous manipulation.

30. The method of claim 14, wherein the steps of the method are performed sequentially.

31. The method of claim 29, wherein the solution comprises one or more of the following: ligase, Uracil-DNA Glycosylase, an apurinic/apyrimidinic endonuclease, an exonuclease, ATP, and dNTPs.

32. The method of claim 14, wherein the oligonucleotide-attached nucleic acid molecule is immobilized on a solid support.

33. The method of claim 32, wherein the molecule is immobilized non-covalently.

34. A kit comprising:
(a) a nucleic acid comprising at least one cleavable base;
(b) a uracil-DNA glycosylase;
(c) an apurinic/apyrimidinic endonuclease; and
(d) an exonuclease.

35. The kit of claim 34, wherein the nucleic acid comprises a degradable adaptor.

36. The kit of claim 35, wherein the degradable adaptor is a partially double-stranded oligonucleotide adaptor, a double-stranded oligonucleotide adaptor, or a stem-loop oligonucleotide adaptor.

37. The kit of claim 36, wherein the stem-loop oligonucleotide adaptor comprises:
(a) a 5′ segment comprising at least one cleavable base;
(b) an intermediate segment coupled to the 3′-end of the 5′ segment; and
(c) a 3′ segment coupled to the 3′-end of the intermediate segment, wherein the 5′ segment and 3′ segment are at least 80% complementary.

38. The kit of claim 37, wherein the 3′ segment does not contain a cleavable base.

39. The kit of claim 37, wherein the intermediate segment comprises at least one cleavable base.

40. The kit of claim 39, wherein the 5′ segment and the intermediate segment of the stem-loop oligonucleotide adaptor comprises a cleavable base every 3-6 bases.

41. The kit of any one of claims 34 and 37-40, wherein the cleavable base is deoxyuridine.

42. The kit of claim 34, wherein the apurinic/apyrimidinic endonuclease is APE 1.

43. The kit of claim 34, wherein the exonuclease is Exonuclease I.
